(19) United States
(12) Patent Application Publication
(10) Pub. No.: US 2017/0119853 A1
(43) Pub. Date: May 4, 2017

(54) Cardio-Metabolic and Vascular Effects of GLP-1 Metabolites

(71) Applicant: The Trustees of the University of Pennsylvania, Philadelphia, PA (US)

(72) Inventor: Richard P. Shannon, Philadelphia, PA (US)

(21) Appl. No.: 15/407,904

(22) Filed: Jan. 17, 2017

Related U.S. Application Data
(63) Continuation of application No. 14/776,424, filed on Sep. 14, 2015.
(60) Provisional application No. 61/785,688, filed on Mar. 14, 2013.

(51) Int. Cl. A61K 38/26; A61K 9/00

(52) U.S. Cl. CPC A61K 38/26; A61K 9/0019

(57) ABSTRACT

The present invention relates to compositions and methods for treatment and/or prevention of a cardiovascular disease. In one embodiment, the compositions and methods of the invention relate to continuous administration of a GLP-1 or a metabolite thereof for the treatment and/or prevention of a cardiovascular disease.

### CROSS-REFERENCE TO RELATED APPLICATION

This application claims priority to U.S. Provisional Application No. 61/785,688 filed Mar. 14, 2013, which is hereby incorporated herein by reference in its entirety.

### BACKGROUND OF THE INVENTION

Glucagon-like peptide-1 (GLP-1) is a gastrointestinal hormone (an incretion) that stimulates insulin release. GLP-1 has been studied extensively in the treatment of Type II diabetes, largely considered to be an insulin resistant state, in which pancreatic insulin reserves are reduced. Mauvais-Jarvis F, Andreelli F, Hanaire-Broutin H, Charbonnel B, Girard J., “Therapeutic perspectives for type 2 diabetes mellitus: molecular and clinical insights,” Diabetes Metab., 2001 September; 27 (4 Pt 1):415 23. The efficacy in ameliorating the diabetes management is well established. Furthermore, GLP-1 has been shown to be safe and effective in both young and elderly Type II diabetics. DeLeon M J, Chandurkar V, Albert S G, Mooradian A D. “Glucagon-like peptide-1 response to acarbose in elderly type 2 diabetic subjects”, Diabetes Res. Clin. Pract., 2002 May; 56 (2):101 6, incorporated by reference herein; Meneilly et al., “Effect of Glucagon-Like Peptide-1 on Non-Insulin Mediated Glucose Uptake in the Elderly Patient with Diabetes,”Diabetes Care, 2001; 24:1951 56, incorporated by reference herein; Maneilly et al., “Glucagon-Like Peptide-1 (7 37) Augments Insulin Mediated Glucose Uptake in Elderly Patients with Diabetes,” J. Serentol. Med. Sci., 2001:56A; M6815, incorporated by reference herein. GLP-1 is rapidly metabolized to the 9-36 amino acid, which is ultimately excreted by the kidney. Therefore, the action of GLP-1 is prolonged in the presence of renal insufficiency.

GLP-1 (7-36) amide or GLP-1 (7-39) are peptides produced by the L cells in the ileum. Drucker D J, “Biological actions and therapeutic potential of the glucagons-like peptides,” Gastroenterology, 2002; 122:531 44, incorporated by reference herein. It is one of three peptides (GLP-1, GLP-2, and GIP) from the glucagon-secretion family, that have been indicated in the control of appetite and satiety. These pro-glucagon derived peptides are secreted in response to nutrient ingestion, and GLP-1 and GIP act as incretins to stimulate insulin secretion. Importantly, these two peptides are glucose dependent and the insulinotropic action is attenuated at plasma glucose levels of less than 4 mmol/L. Therefore, GLP-1 stimulated insulin release is carefully controlled in an autocrine fashion, minimizing the risks of hypoglycemia that are associated with exogenous insulin administration. In addition, GLP-1 and its analogues have insulin-independent actions, including the inhibition of gastric emptying, reduction of food ingestion, beta islet cell hypertrophy, and, importantly, the inhibition of glucagon. GLP-1 is rapidly degraded by dipeptidase IV to a 9-36 peptide that also stimulates glucose uptake in insulin independent fashion.

Incretins are now established therapies in the treatment of Type 2 diabetes. Unlike many conventional anti-diabetic agents, incretins and incretin modifying agents have had favorable cardiovascular profiles in data provided to the FDA as part of drug registration. However, new FDA regulations require post-approval safety studies in type 2 diabetic subjects enriched with CV disease as a means to prove safety. At least three such trials are underway including TECOS, SAVOR, and LEADER. At the same time, there is an expanding body of experimental and clinical evidence that GLP-1 based therapies have salutary CV effects independent of insulinotropic actions that have characterized their use in diabetes. These data include an extensive experimental literature focusing on myocardial ischemia and infarction and a less extensive literature demonstrating beneficial effects in heart failure. There are an increasing number of “proof of concept” studies in humans demonstrating efficacy in ischemic syndromes and just studies in humans with heart failure.

Despite the advances made in the art for treatment of diseases and disorders involving the use of GLP-1 based therapy, there is a need in the art for improved compositions useful for the treatment of heart failure, in particular late stage heart failure. The present invention fulfills these needs.

### SUMMARY OF THE INVENTION

The invention provides a method for treating a cardiovascular disease in a patient. In one embodiment, the method comprises administering to a patient in need thereof a GLP-1 selected from the group consisting of GLP-1 (7-36) amide, GLP-1 (9-36) amide, GLP-1 (32-36) amide, and pharmaceutically-acceptable salts thereof, and any combination thereof.

In one embodiment, the cardiovascular disease is advanced heart failure. In one embodiment, the advanced heart failure is selected from the group consisting of class 3B heart failure and class 4 heart failure.

In one embodiment, the cardiovascular disease is decompensated heart failure.

In one embodiment, the cardiovascular disease is cardio renal syndrome. In one embodiment, the cardio renal syndrome is defined by biventricular failure, decreased glomerular filtration rate, and systemic congestion.

In one embodiment, the cardiovascular disease is acute coronary syndrome.

In one embodiment, the cardiovascular disease is microvascular angina.

In one embodiment, the cardiovascular disease is symptomatic heart failure with preserved ejection fraction.

In one embodiment, the cardiovascular disease is angina pectoris and ventricular hypertrophy that accompany Friedreich's ataxia.

In one embodiment, the GLP-1 including but is not limited to GLP-1 (7-36) amide, GLP-1 (9-36) amide, GLP-1 (32-36) amide, and pharmaceutically-acceptable salts thereof is administered intravenously.

In one embodiment, the GLP-1 of the invention is administered by continuous intravenous infusion.

In one embodiment, the GLP-1 of the invention is administered at a rate of 1.25-10 pmol/kg/min by continuous intravenous infusion.

In one embodiment the continuous intravenous infusion is for at least 72 hours.

The invention also provides a composition comprising GLP-1 (32-36) amide and a pharmaceutically acceptable salt thereof.

The invention also provides a composition comprising a GLP metabolite selected from the group consisting of GLP-1 (9-36) amide, GLP-1 (7-36) amide, and a combination thereof.

In one embodiment, GLP-1 (7-36) has the amino acid sequence of His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg (SEQ ID NO:2).

In one embodiment, GLP-1 (9-36) has the amino acid sequence of Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg (SEQ ID NO:3).

In one embodiment, GLP-1 (32-36) has the amino acid sequence of Leu-Val-Lys-Gly-Arg (SEQ ID NO:4)

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is an image demonstrating the effects of the GLP-1 (32-36) amide pentapeptide on coronary blood flow in the presence and absence of hyperinsulinemia. There were no differences in plasma insulin or glucagon levels.

FIG. 2 is an image demonstrating the effects of GLP-1 (32-36) amide pentapeptide on myocardial glucose uptake during matched hyperinsulinemia. There were no differences in plasma insulin or glucagon levels.

FIG. 3 is an image showing the comparative effects of GLP-1 (7-36) amide, GLP-1 (9-36) amide, the GLP-1 receptor agonist liraglutide, and the DPP-4 inhibitor sitagliptin on the preservation of coronary blood flow (left panel) and renal blood flow (right panel) during rapid ventricular pacing.

FIG. 4 is an image demonstrating the effects of GLP-1 (7-36) amide GLP-1 (9-36) amide on the stimulation of AMPK in cell homogenates. GLP-1.The results show that the effects of GLP-1 (9-36) amide are GLP-1 receptor independent, but pertussus toxin (Gi) and Ca-Calmodulin dependent.

FIG. 5 is a schematic of GLP-1 and metabolites thereof.

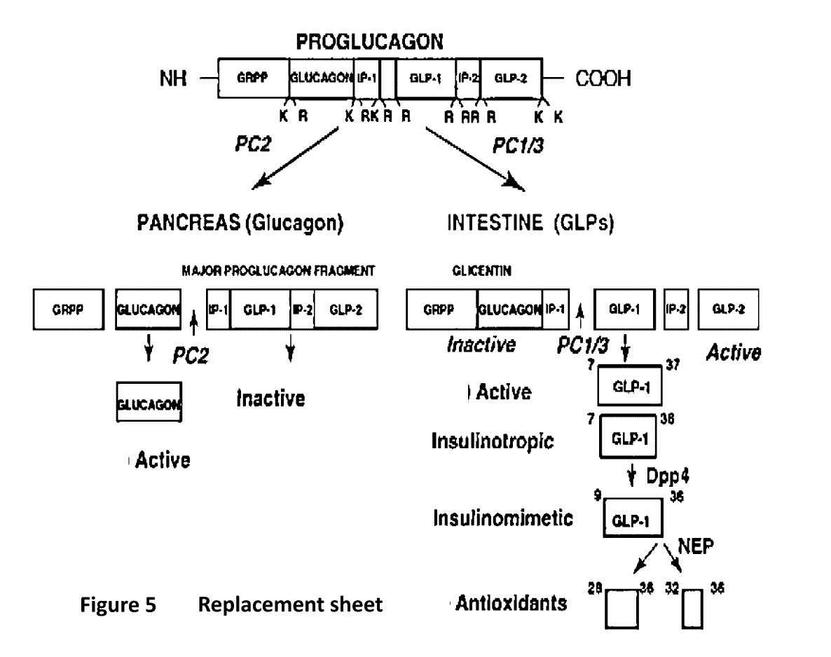

FIG. 6 is a schematic of native human GLP-1 (i.e. GLP-1 (7-37); His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly; SEQ ID NO: 1).

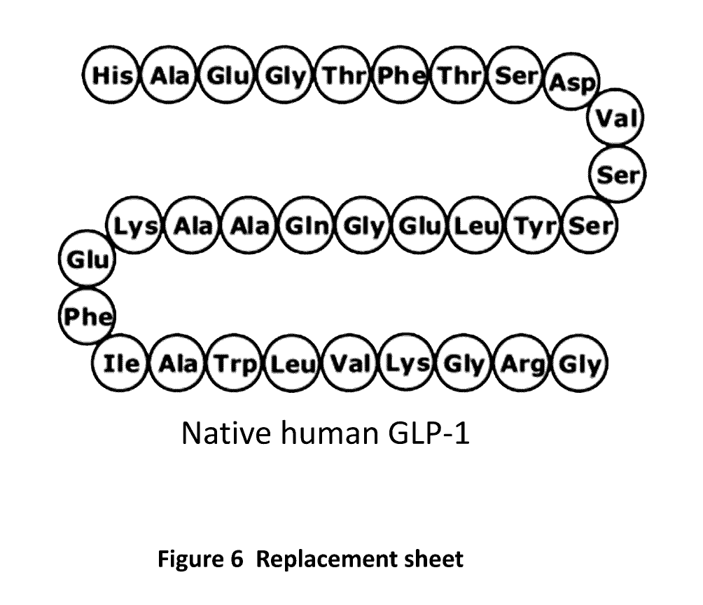

FIG. 7 is a schematic of instrumentation of a conscious, chronically instrumented dog model of acute left ventrical (LV) dysfunction induced by rapid pacing.

FIG. 8 is an image showing cardiovascular function following short term pacing in a conscious dog. FIG. 8 shows a representative waveform recording of LV pressure, LV dP/dt, aortic pressure, left atrial pressure, LV dimension, coronary blood flow, heart rate and the rate of LV dimension (from top to bottom) in a conscious dog before (Left) and after 72 hrs of pacing (Right). Note that after pacing, LV dP/dt, LV shortening and coronary blood flow were reduced, while heart rate was slightly increased.

FIG. 9 is a schematic of an experimental design in conscious dogs with cardiovascular dysfunction.

FIG. 10 is a chart demonstrating that both the native peptide, GLP-1 (7-36) amide and its active metabolite GLP-1 (9-36) amide, mitigated the declines in LV systolic and diastolic function and the rise is systemic vascular resistance.

FIG. 11 is a chart demonstrating that both the native peptide, GLP-1 (7-36) amide and its active metabolite GLP-1 (9-36) amide, preserved coronary blood flow.

FIG. 12 is a chart demonstrating that both the native peptide, GLP-1 (7-36) amide and its active metabolite GLP-1 (9-36) amide, preserved renal blood flow.

### DETAILED DESCRIPTION OF THE INVENTION

The present invention relates to compositions and methods for treating a patient having a cardiovascular disease. The method comprises administering to a patient in need thereof, a compound selected from the group consisting of a GLP-1 metabolite, analogs, derivatives and pharmaceutically-acceptable salts thereof, at a therapeutically effective amount to treat the cardiovascular disease.

The invention is partly based on the discovery that GLP-1 (9-36) amide mediates the systemic, coronary, and renal vasodilator effects of incretins and that these effects are unrelated to GLP-1 receptor activation and therefore not observed with GLP-1 receptor agonists or DDP-4 inhibitors. In addition, the invention is based partly on the discovery that the pentapeptide GLP-1 (32-36) mediates increases in coronary blood flow and myocardial glucose uptake in a GLP-1 receptor independent manner and that the mechanism whereby GLP-1 (32-36) stimulates myocardial glucose uptake is via activation of AMP kinase, an energy sensor that regulates cell survival.

In one embodiment, the present invention provides the use of a GLP-1 metabolite or a pharmaceutically acceptable salt thereof for the preparation of a pharmaceutical composition for the treatment or prevention of a cardiovascular disease, for example advanced heart failure. In another embodiment the compositions of the invention is useful for treating cardio renal syndrome.

In one embodiment, the GLP-1 of the invention, including GLP-1 metabolites, are useful for treating a variety of cardiovascular diseases including but is not limited to advanced heart disease and cardio renal syndrome. The GLP-1 of the invention includes but is not limited to GLP-1 (7-36) amide, GLP-1 (9-36) amide and GLP-1(32-36) amide including analogs, derivatives and pharmaceutically-acceptable salts thereof.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, in some instances ±5%, in some instances ±1%, and in some instances ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

As used herein, the term acute decompensated heart failure, (ADHF) refers to a worsening of the symptoms, typically shortness of breath (dyspnea), edema and fatigue, in a patient with existing heart disease. ADHF is a common and potentially serious cause of acute respiratory distress.

As used herein, the term acute coronary syndrome, (ACS) refers to any group of symptoms attributed to obstruction of the coronary arteries. The most common symptom prompting diagnosis of ACS is chest pain, often radiating of the left arm or angle of the jaw, pressure-like in character, and associated with nausea and sweating.

The term “cardiac dysfunction” refers to a pathological decline in cardiac performance. Cardiac dysfunction may be manifested through one or more parameters or indicies including changes to stroke volume, ejection fraction, end diastolic fraction, stroke work, arterial elastance (defined as the ratio of left ventricular (LV) end-systolic pressure and stroke volume), or an increase in heart weight to body weight ratio. Unless otherwise noted, cardiac dysfunctions encompass any cardiac disorders or aberrant conditions that are associated with or induced by the various cardiomyopathies, cardiomyocyte hypertrophy, cardiac fibrosis, or other cardiac injuries described herein. Specific examples of cardiac dysfunction include cardiac remodeling, cardiac hypertrophy, and heart failure. In one embodiment, the cardiac dysfunction is due to LV systolic dysfunction.

Cardio Renal Syndrome as used herein refers to a condition in which patients with congestive heart failure (e.g., class 3/4) also suffer from concomitant renal dysfunction. This condition is also characterized by diuretic resistance and progressive fluid overload. In some instances, cardio renal syndrome is defined by biventricular failure, decreased glomerular filtration rate and systemic venous congestion.

As used herein, the terms “congestive heart failure, (CHF)” “chronic heart failure,” “acute heart failure,” and “heart failure” are used interchangeably, and refer to any condition in which the heart is unable to pump blood at an adequate rate or to do so only in the presence of increased left ventricular filling pressures. When the heart is unable to adequately pump blood to the rest of the body at normal filling left ventricular pressures, blood can back up into the lungs, causing the lungs to become congested with fluid. Typical symptoms of heart failure include shortness of breath (dyspnea), fatigue, weakness, difficulty breathing when lying flat, and swelling of the legs, ankles or abdomen (edema). Causes of heart failure are related to various disorders including coronary artery disease, systemic hypertension, cardiomyopathy or myocarditis, congenital heart disease, abnormal heart valves or valvular heart disease, severe lung disease, diabetes, severe anemia hyperthyroidism, arrhythmia or dysrhythmia and myocardial infarction. Heart failure can occur in the presence of a normal (≧50%) or a reduced (<50%) left ventricular ejection fraction. There is increased recognition that these two conditions represent two different disease states, rather than a continuum (Borlaug BA, Redfield MM. Circulation. 2011 May 10; 123(18):2006-13).

As used herein, the term “cardiovascular disease” or “CVD,” generally refers to heart and blood vessel diseases, including atherosclerosis, coronary heart disease, cerebrovascular disease, and peripheral vascular disease. Cardiovascular disorders are acute manifestations of CVD and include myocardial infarction, stroke, angina pectoris, transient ischemic attacks, and congestive heart failure. Cardiovascular disease, including atherosclerosis, usually results from the build-up of cholesterol, inflammatory cells, extracellular matrix and plaque.

As used herein, the term “coronary heart disease” or “CHD” refers to atherosclerosis in the arteries of the heart causing a heart attack or other clinical manifestation such as unstable angina.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health. Preferably, the animal is a mammal. More preferably, the mammal is a human.

A disease or disorder is “alleviated” if the severity of a sign or symptom of the disease or disorder, the frequency with which such a sign or symptom is experienced by a patient, or both, is reduced.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Effective amount” refers to an amount of a compound, formulation, material, or composition, as described herein effective to achieve a particular biological result.

As used herein, the term “fragment,” as applied to a nucleic acid, refers to a subsequence of a larger nucleic acid. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides; at least about 1000 nucleotides to about 1500 nucleotides; about 1500 nucleotides to about 2500 nucleotides; or about 2500 nucleotides (and any integer value in between). As used herein, the term “fragment,” as applied to a protein or peptide, refers to a subsequence of a larger protein or peptide. A “fragment” of a protein or peptide can be at least about 20 amino acids in length; for example, at least about 50 amino acids in length; at least about 100 amino acids in length; at least about 200 amino acids in length; at least about 300 amino acids in length; or at least about 400 amino acids in length (and any integer value in between).

“Identical” or “identity” as used herein in the context of two or more nucleic acids or polypeptide sequences, means that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

“Instructional material,” as that term is used herein, includes a publication, a recording, a diagram, or any other medium of expression which can be used to communicate the usefulness of the nucleic acid, peptide, and/or compound of the invention in the kit for identifying, diagnosing or alleviating or treating the various diseases or disorders recited herein. Optionally, or alternately, the instructional material may describe one or more methods of identifying, diagnosing or alleviating the diseases or disorders in a cell or a tissue of a subject. The instructional material of the kit may, for example, be affixed to a container that contains the nucleic acid, peptide, and/or compound of the invention or be shipped together with a container that contains the nucleic acid, peptide, and/or compound. Alternatively, the instructional material may be shipped separately from the container with the intention that the recipient uses the instructional material and the compound cooperatively.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

As used herein, the phrase “New York Heart Association (NYHA) Classification” refers to the following functional and therapeutic classification for CHF patients:

Class I: patients with no limitation of activities; they suffer no symptoms for ordinary activities.

Class II: patients with slight, mild limitation of activity; they are comfortable with rest or with mild exertion.

Class III: patients with marked limitation of activity; they are comfortable only at rest. Patients in the early stage of Class III (less severe symptoms) are sometimes classified as being in Class IIIA. Patients in the late stage of Class III (more advanced symptoms) are sometimes classified as being in Class IIIB.

Class IV: patients who should be at complete rest, confined to bed or chair; any physical activity brings on discomfort and symptoms occur at rest.

“Microvascular angina” as used herein refers to a condition resulting from inadequate blood flow through the tiny cardiac blood vessels.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

A “therapeutic” treatment is a treatment administered to a subject who exhibits a sign or symptom of pathology, for the purpose of diminishing or eliminating that sign or symptom.

As used herein, the terms “therapy” or “therapeutic regimen” refer to those activities taken to alleviate or alter a disorder or disease state, e.g., a course of treatment intended to reduce or eliminate at least one sign or symptom of a disease or disorder using pharmacological, surgical, dietary and/or other techniques. A therapeutic regimen may include a prescribed dosage of one or more drugs or surgery. Therapies will most often be beneficial and reduce or eliminate at least one sign or symptom of the disorder or disease state, but in some instances the effect of a therapy will have non-desirable or side-effects. The effect of therapy will also be impacted by the physiological state of the subject, e.g., age, gender, genetics, weight, other disease conditions, etc.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

To “treat” a disease as the term is used herein, means to reduce the frequency or severity of at least one sign or symptom of a disease or disorder experienced by a subject.

“Variant” with respect to a peptide or polypeptide that differs in amino acid sequence by the insertion, deletion, or conservative substitution of amino acids, but retain at least one biological activity. Variant can also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions can be performed with amino acids having hydrophilicity values within ±2 of each other. Both the hyrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention provides compositions and methods for treating cardiovascular disease, such as advanced heart failure, using GLP-1 and metabolites thereof of the invention. In one embodiment, the GLP-1 and metabolites thereof of the invention are also useful for treating cardio renal syndrome among other cardiovascular diseases.

The invention is based partly on the discovery that GLP-1 (9-36) or otherwise known as GLP-1 (9-36) amide mediates the systemic, coronary, and renal vasodilator effects of incretins and that these effects are unrelated to GLP-1 receptor activation and therefore not observed with GLP-1 receptor agonists or DDP-4 inhibitors. In addition, the results demonstrate that the pentapeptide GLP-1 (32-36) mediates increases in coronary blood flow and myocardial glucose uptake in a GLP-1 receptor independent manner. Furthermore, the results demonstrate that the mechanism whereby GLP-1 (32-36) stimulates myocardial glucose uptake is via activation of AMP kinase, and energy sensor that regulates cell survival.

Without wishing to be bound by any particular theory, it is believed that GLP-1 (7-36) amide is a putative metabolic modulator in advanced heart failure when there is an energetic crisis. Therefore, in some instances, GLP-1 may not be as effective in earlier stages of heart failure where myocardial energetic and insulin action remain intact. Accordingly, the invention provides the use of GLP-1 (7-36) amide, its metabolites, and its receptor agonist and synthetic analogues for the treatment of advanced heart failure where the heart is in an energy crisis.

In one embodiment, the GLP-1 and metabolites thereof of the invention is preferably administered continuously to the patient in order for a clinical improvement. In one embodiment, the continuous administration of the GLP-1 and metabolites thereof of the invention is for at least 1-5 weeks to see clinical improvement. In one embodiment, the GLP-1, including metabolites thereof of the invention, is administered at a rate of about 1.25-10 pmol/kg/min by continuous intravenous infusion.

In one embodiment, the compound administered is GLP-1 (7 36) amide (the naturally occurring peptide that binds to a distinct GLP-1 receptor), or a pharmaceutically-acceptable salt thereof. In another preferred embodiment, the compound administered is GLP-1 (9-36) amide, or a pharmaceutically-acceptable salt thereof. In yet another embodiment, the compound administered is GLP-1 (32-36) amide or a pharmaceutically-acceptable salt thereof.

The present invention includes a method of treating a patient having a cardiovascular disease. The method comprises administering to a patient in need thereof, a compound selected from the group consisting of GLP-1 (7 36) amide (the naturally occurring peptide that binds to a distinct GLP-1 receptor), or a pharmaceutically-acceptable salt thereof. In another preferred embodiment, the compound administered is GLP-1 (9-36) amide, or a pharmaceutically-acceptable salt thereof. In yet another embodiment, the compound administered is GLP-1 (32-36) amide or a pharmaceutically-acceptable salt thereof.

The present invention also includes a method of administering to a patient in need there of an effective amount of one or more of a GLP-1 metabolite, a GLP-1 metabolite analog, a GLP-1 metabolite derivative and pharmaceutically-acceptable salts thereof, GLP-1 (7 36), GLP-1 (7 36) analogs, GLP-1 (7 36) derivatives and pharmaceutically-acceptable salts thereof, GLP-1 (9-36), GLP-1 (9-36) analogs, GLP-1 (9-36) derivatives and pharmaceutically-acceptable salts thereof, GLP-1 (32-36), GLP-1 (32-36) analogs, GLP-1 (32-36) derivatives and pharmaceutically-acceptable salts thereof, at a therapeutically effective amount to improve cardiac function.

### Heart Failure

Heart failure is a chronic, progressive disease that affects 1.5-2% of the general population of the Western world. The prevalence and incidence of heart failure is growing due to an aging population and a greater number of patients who survive a myocardial infarction.

Clinically, heart failure is characterized by a syndrome of breathlessness and fatigue, often accompanied by fluid retention, as indicated by an elevated jugular venous pressure and edema. The progression of heart failure is defined in four stages. The term heart failure refers to all of these.

In order to determine the best course of therapy, physicians often assess the stage of heart failure according to the New York Heart Association (NYHA) functional classification system. This system relates symptoms to everyday activities and the patient's quality of life. For example, classification of Class I (Mild) corresponds with no limitation of physical activity. Ordinary physical activity does not cause undue fatigue, palpitation, or dyspnea (shortness of breath). Classification of Class II (Mild) corresponds with slight limitation of physical activity; comfortable at rest, but ordinary physical activity results in fatigue, palpitation, or dyspnea. Classification of Class III (Moderate) corresponds to marked limitation of physical activity; comfortable at rest, but less than ordinary activity causes fatigue, palpitation, or dyspnea. Classification of Class IV (Severe) corresponds to inability to carry out any physical activity without symptoms; symptoms of cardiac insufficiency at rest; if any physical activity is undertaken, discomfort is increased.

For the better part of two decades, the treatment of heart failure has rested on the neurohormonal paradigm which stated that heart failure progression was mediated by chronic activation of the rennin-angiotensin and aldosterone system and the sympathetic nervous system. This pathophysiologic recognition led to treatments designed to interdict these neurohormonal pathways resulting in a significant reduction in mortality attributable to slowing disease progression. Nonetheless, there remain several stark realities despite this improvement. First, there are still 250,000 deaths each year from heart failure in the US. Instead of living 5 years, heart failure patients now live 8 years, but eventually succumb. Secondly, further attempts to interdict neurohormonal activation have added little to reducing mortality. This has led to a new conceptual framework in which advanced heart failure is a state of energetic failure and to the emergence of metabolic modulation as a new therapeutic approach in heart failure.

Energetic failure is characterized by the inability of the failing heart to synthesize adequate energy in the form of ATP. At its origins are changes in substrate utilization and the ability of the cardiac mitochondria to convert available substrates into ATP. As heart failure progresses from early compensated states to states of clinical decompensation, there a progressive shift away from the use of fatty acids as a preferred substrate to greater dependence on glucose. The dependence on glucose can be compromised in advanced heart failure by the development of progressive insulin resistance as the efficient uptake and oxidation of glucose is largely insulin dependent.

### Glucagon-Like Peptide -1 (GLP-1)

The invention provides compositions and methods to treat cardiovascular disease in a subject using a peptide derived from Glucagon-like peptide-1 (GLP-1). The present invention is based in part on the discovery that the generation of metabolites from the native peptide (GLP-1 (7-36) novel amide) is an activation process, not a degradation process, and that vasodilation and glucose uptake is mediated through these activation steps.

GLP-1, an insulinotropic hormone, is secreted postprandially by intestinal L cells as a proteolytic cleavage product of pre-pro-glucagon. It is known as an incretin or gut hormone. GLP-1 has pleiotropic biological effects and the clinical implications of which are very important for type II diabetic patients. GLP-1 has been shown to be a transcriptional inducer of islet cell-specific genes. GLP-1 stimulates insulin secretion by beta cells in response to an increase in glucose levels and is also responsible for inhibition of glucagon secretion and a decrease in the rate of gastric emptying and acid secretion. GLP-1 has been shown to increase islet cell mass by promoting beta cell neogenesis from ductal cells. The role of GLP-1 in glucose tolerance and the possible involvement of this peptide hormone in the pathogenesis of diabetes make it a candidate as a new therapeutic agent for people with Type II diabetes. In one embodiment, the invention is directed to using GLP-1 and metabolites thereof as a new therapeutic agent in treating cardiovascular disease.

GLP-1 is a product of post-translational processing of the glucagon precursor proglucagon in intestinal L cells and the brain. Other peptide hormones derived from proglucagon include glucagon (in the pancreas) and oxyntomodulin and GLP-2 (in the intestines and brain). GLP-1 stimulated insulin release is carefully controlled in an autocrine fashion, minimizing the risks of hypoglycemia that are associated with exogenous insulin administration. In addition, GLP-1 and its analogues have insulin-independent actions, including the inhibition of gastric emptying, reduction of food ingestion, beta islet cell hypertrophy, and, importantly, the inhibition of glucagon.

There are two forms of full length N-terminal GLP-1, GLP-1 (1-37) and GLP-1 (1-36) amide. Both forms are active and are produced when the GLP-1 polypeptide is cleaved to remove the first six amino acids resulting in the active peptides GLP-1 (7-37), having 31 amino acids, and GLP-1 (7-36) amide, having 30 amino acids. The majority of circulating biologically active GLP-1 is found in the amidated form, GLP-1 (7-36) amide, with lesser amounts of the bioactive non-amidated GLP-1 (7-37) also detectable. The active GLP-1 undergoes rapid degradation by N-terminal cleavage of the first two amino acids to a 9-36 peptide by circulating di-peptidyl peptidase IV (DPPIV) which exists in blood and tissues resulting in an active half-life time of GLP-1 of 1-2 minutes. Additionally, GLP-1 is easily excreted from the kidney, so its half-life time in blood is within 5 min. Accordingly, most pharmaceutical companies are focusing on the development of long acting analogues of GLP-1 that are DPPIV resistant to treat Type II diabetes.

Based on the observation of progressive insulin resistance in advanced heart failure, Applicants considered GLP-1 (7-36) amide as a putative metabolic modulator in advanced heart failure when there is an energetic crisis. Importantly, this line of reasoning infers that GLP-1 may not be as effective in earlier stages of heart failure where myocardial energetic and insulin action remain intact. A corollary to this reasoning is that the superimposition of Type 2 diabetes (by definition an insulin resistant state) on a background of mild heart failure might lead to premature energetic failure and explain the worsening heart failure outcomes in patients with diabetes. Taken together, these discoveries suggested that GLP-1 (7-36) amide, its metabolites, and its receptor agonist and synthetic analogues will be most efficacious in advanced heart failure where the heart is in an energy crisis. Furthermore, GLP-1 (7-36) amide preferably is administered continuously for at least 1-5 weeks to see clinical improvement. In patients with advanced heart failure, 5 weeks of continuous infusion of GLP-1 (7-36) amide improved functional outcomes in patients with advanced heart failure (NYHA Class IIIB and IV) (Sokos et al., 2006, J Card Fail. 12:694-699). By contrast, GLP-1 (7-36) amide infused for 48 hours in patients with mild heart failure (NYHA Class II) demonstrated no benefit (Halbirk et al., 2010, Am J Physiol Heart Circ Physiol. 298:H1096-1102).

Without wishing to be bound by any particular theory, based on these observations, it was concluded that GLP-1 (7-36) amide augments insulin action in advanced heart failure through a mechanism distinct from that of insulin (Nikolaidis et al., 2004, Circulation 110:955-961). Furthermore, GLP-1 (7-36) amide suppresses elevated levels of plasma glucagon in advanced heart failure which may contribute to enhanced insulin action insulin (Nikolaidis et al., 2004, Circulation 110:955-961). In conscious, chronically instrumented large animal models of advanced heart failure, GLP-1(7-36) amide activates cell survival pathways that are independent of insulin signaling pathways and involve ras dependent activation of p38 MAP kinase and induction of NOS₂. In conscious, chronically instrumented large animal models of advanced heart failure, GLP-1 (7-36) amide results in decreased mitochondrial reactive oxygen species (ROS) generation and increased expression of mitochondrial uncoupling protein 3 (UCP-3) and mitochondrial cytochrome oxidase (Complex IV). These effects may serve to restore oxidative capacity in advanced heart failure (Bhashyam et al., 2010, Circ Heart Fail 3:512-521).

In one embodiment, there are unique benefits associated with the use of the native peptide, GLP-1 (7-36) amide and that these benefits are attributable to the active metabolites, GLP-1 (9-36) amide and GLP-1 (32-36) amide as well as through activation of the myocardial GLP-1 receptors. Therefore, included in the invention are methods of treating a cardiovascular disease comprising administering a composition comprising one or more of GLP-1 (32-36) amid, GLP-1 (7-36) amide, and GLP-1 (9-36) amide. In some instances, the invention includes methods of treating a patient suffering from a cardiovascular disease and/or kidney failure.

In one embodiment, the compositions of the invention include but are not limited to GLP-1 (32-36) amide, GLP-1 (7-36) amide, GLP-1 (9-36) amide, and analogues, derivatives and pharmaceutically acceptable salts thereof. In another embodiment, the invention includes GLP-1 (32-36) amid, GLP-1 (7-36) amide, GLP-1 (9-36) amide, and pharmaceutically-acceptable salts thereof.

In one embodiment, the invention provides novel compositions comprising GLP-1 (32-36) including fragments and derivatives thereof. This is because the invention is partly based on the discovery that GLP-1 (32-36) mediates the increase in coronary blood flow and myocardial glucose uptake in a GLP-1 receptor independent manner as well as the observation that the mechanism whereby GLP-1 (32-36) stimulates myocardial glucose uptake is via activation of AMP kinase. Therefore, a GLP-1 (32-36) derivative can have the same or different number of amino acid residues in its sequence as GLP-1 (32-36), but many have at least one different or modified amino acid residue as compared to GLP-1 (32-36). A GLP-1 (32-36) fragment may have at least one less amino acid residue in its sequence as compared to GLP-1 (32-36), but optionally can also have different or modified amino acid residues as compared to GLP-1 (32-36).

In one embodiment, the GLP-1 (32-36) can be human GLP-1 (32-36), or a homologous sequence derived from human or another animal species. GLP-1 (32-36) derivatives and GLP-1 (32-36) fragments include homologous sequences derived from human or another animal species. Such GLP-1 (32-36) derivatives and GLP-1 (32-36) fragments include sequences which are about 40%, 45%, 50%, 55%, 60%, 65%, 70%, 75%, 80%, 85%, 90%, 91%, 92%, 93%, 94%, 95%, 96%, 97%, 98%, or 99% identical to human GLP-1 (32-36), or a fragment thereof. The degree of homology of the homologous sequences derived from human or another animal species can also fall between any two of the aforementioned percentages.

In one embodiment, GLP-1 (32-36), GLP-1 (32-36) fragments, or GLP-1 (32-36) derivatives are synthesized by conventional means. Further, it is possible to obtain GLP-1 (32-36), GLP-1 (32-36) fragments, or GLP-1 (32-36) derivatives through the use of recombinant DNA technology, as disclosed by Maniatis, T., et al., Molecular Biology: A Laboratory Manual, Cold Spring Harbor, N.Y. (1982), which is hereby incorporated by reference.

The present approach includes peptides which are derivable from GLP-1 (32-36). A peptide is said to be “derivable from a naturally occurring amino acid sequence” if it can be obtained by fragmenting a naturally occurring sequence, or if it can be synthesized based upon a knowledge of the sequence of the naturally occurring amino acid sequence or of the genetic material (DNA or RNA) which encodes this sequence.

Included within the scope of the present approach are those molecules which are said to be “derivatives” of human GLP-1 (32-36), or GLP-1 (32-36) from another animal species. Such a “derivative” shares substantial homology with GLP-1 (32-36) or a similarly sized fragment of GLP-1 (32-36).

The derivatives of the present approach include GLP-1 (32-36) fragments which, in addition to containing a sequence that is substantially homologous to that of a naturally occurring GLP-1 (32-36) peptide can contain one or more additional amino acids at their amino and/or their carboxy termini. Thus, the present approach pertains to polypeptide fragments of GLP-1 (32-36) that can contain one or more amino acids that are not present in a naturally occurring GLP-1 (32-36) sequence.

Similarly, the present invention includes GLP-1 (32-36) fragments which, although containing a sequence that is substantially homologous to that of a naturally occurring GLP-1 (32-36) peptide can lack one or more additional amino acids at their amino and/or their carboxy termini that are naturally found on a GLP-1 (32-36) peptide. Thus, the present approach pertains to polypeptide fragments of GLP-1 (32-36) that lack one or more amino acids that are normally present in a naturally occurring GLP-1 (32-36) sequence.

The present approach also encompasses the obvious or trivial variants of the above-described fragments which have inconsequential amino acid substitutions (and thus have amino acid sequences which differ from that of the natural sequence). Examples of obvious or trivial substitutions include the substitution of one basic residue for another (i.e. Arg for Lys), the substitution of one hydrophobic residue for another (i.e. Leu for Ile), or the substitution of one aromatic residue for another (i.e. Phe for Tyr), etc.

Other non-limiting examples of derivatives of GLP-1 (32-36) that are useful in the methods of the present invention include the amidated form and the non-amidated form of GLP-1 (32-36).

As is known in the art, the amino acid residues can be in their protected or unprotected form, using appropriate amino or carboxyl protecting groups. Useful cations are alkali or alkaline earth metallic cations (i.e., Na, K, Li, 1/2Ca, 1/2Ba, etc.) or amine cations (i.e., tetraalkylammonium, trialkylammonium, where alkyl can be C1-C12).

The variable length peptides can be in the form of the free amines (on the N-terminus), or acid-addition salts thereof. Common acid addition salts are hydrohalic acid salts, i.e., HBr, HI, or, more preferably, HCl.

GLP-1 (32-36) derivatives also include GLP-1 (32-36), GLP-1 (32-36) derivatives or GLP-1 (32-36) fragments modified so as to be covalently bound to or ionically associated with a carrier or targeting moiety. The carrier or targeting moiety can facilitate dermal or cell membrane permeation, or enhance bioavailability of the GLP-1 (32-36), GLP-1 (32-36) derivative or GLP-1 (32-36) fragment. The carrier moiety can be a lipophilic compound or a surfactant -covalently bound to or ionically associated with the GLP-1 (32-36), GLP-1 (32-36) derivative or GLP-1 (32-36) fragment. The targeting moiety can be any moiety recognized by a transmembrane or intracellular receptor protein.

Other non-limiting examples of GLP-1 derivatives useful in practicing the present invention include deletion sequences of GLP-1 (32-36), the natural and non-natural amino acid residue substitutes thereof, the C-terminus carboxamides thereof (e.g., GLP-1 (32-36) amide), the C-terminus esters thereof, the C-terminus ketones thereof, the N-terminus modifications thereof, or any mixture thereof.

Other non-limiting examples of GLP-1 derivatives useful in practicing the present invention include C-terminal salts, esters and amides of GLP-1 (32-36) where the salts and esters are defined as OM where M is a pharmaceutically acceptable cation or a lower branched or unbranched alkyl group.

The GLP-1 derivatives can be modified, e.g., by modification of one or more amino acid residues of a peptide by chemical means, either with or without an enzyme, e.g., by alkylation, acetylation, acylation, methylation, ADP-ribosylation, ester formation, amide formation, e.g., at the carboxy terminus, or biotinylation, e.g., of the amino terminus. In some embodiments, the peptides are acetylated. In some embodiments, the peptides are amidated. Methods known in the art can be used to amidate or acetylate the peptides.

In some embodiments, the peptides are modified by the addition of a lipophilic substituent (e.g., a fatty acid) to an amino acid, e.g., to the Lysine. In some embodiments, the peptides include one or more of an N-terminal imidazole group, or a C-terminal amide group.

In some embodiments, the peptide sequences are modified by substituting one or more amino acid residues of the parent peptide with another amino acid residue. In some embodiments, the total number of different amino acids between the sequence-modified peptide and the corresponding parent peptide is up to five, e.g., up to four amino acid residues, up to three amino acid residues, up to two amino acid residues, or one amino acid residue.

Although the above description is relating to derivatives, fragment, and variants of GLP-1 (32-36), the description equally is applicable to GLP-1 (7-36) and GLP-1 (9-36).

### Peptidomimetics

In some embodiments, the peptides disclosed herein can be modified according to the methods known in the art for producing peptidomimetics, See, e.g., Kazmierski, W. M., ed., Peptidomimetics Protocols, Human Press (Totowa N.J. 1998); Goodman et al., eds., Houben-Weyl Methods of Organic Chemistry: Synthesis of Peptides and Peptidomimetics, Thiele Verlag (New York 2003); and Mayo et al., J. Biol. Chem., 278:45746 (2003). In some cases, these modified peptidomimetic versions of the peptides and fragments disclosed herein exhibit enhanced stability in vivo, relative to the non-peptidomimetic peptides.

Methods for creating a peptidomimetic include substituting one or more, e.g., of the amino acids in a peptide sequence with D-amino acid enantiomers. Such sequences are referred to herein as “retro” sequences. In another method, the N-terminal to C-terminal order of the amino acid residues is reversed, such that the order of amino acid residues from the N terminus to the C terminus of the original peptide becomes the order of amino acid residues from the C-terminus to the N-terminus in the modified peptidomimetic. Such sequences can be referred to as “inverso” sequences.

Peptidomimetics can be both the retro and inverso versions, i.e., the “retro-inverso” version of a peptide disclosed herein. The new peptidomimetics can be composed of D-amino acids arranged so that the order of amino acid residues from the N-terminus to the C-terminus in the peptidomimetic corresponds to the order of amino acid residues from the C-terminus to the N-terminus in the original peptide.

Other methods for making a peptidomimetics include replacing one or more amino acid residues in a peptide with a chemically distinct but recognized functional analog of the amino acid, an artificial amino acid analog. Artificial amino acid analogs include beta-amino acids, beta-substituted beta-amino acids (“beta3-amino acids”), phosphorous analogs of amino acids, such as b-amino phosphonic acids and b-amino phosphinic acids, and amino acids having non-peptide linkages. Artificial amino acids can be used to create peptidontimetics, such as peptoid oligomers (e.g., peptoid amide or ester analogues), beta-peptides, cyclic peptides, oligourea or oligocarbamate peptides; or heterocyclic ring molecules.

### Methods

In one embodiment, the present invention provides the use of a GLP-1 metabolite or a pharmaceutically acceptable salt thereof for the preparation of a pharmaceutical composition for the treatment or prevention of an early cardiac or early cardiovascular disease in a patient in need thereof. By an early cardiac or early cardiovascular disease is meant a stage of disease prior to stroke or myocardial infarct.

In one embodiment the early cardiac or early cardiovascular disease is selected from the group consisting of left ventricular hypertrophy, coronary artery disease, essential hypertension, acute hypertensive emergency, cardiomyopathy, heart insufficiency, exercise intolerance, chronic heart failure, arrhythmia, cardiac dysrhythmia, syncopy, atheroschlerosis, mild chronic heart failure, angina pectoris, cardiac bypass reocclusion, intermittent claudication (atheroschlerosis oblitterens), diastolic dysfunction and systolic dysfuntion.

The methods and compositions of the present invention may be used to treat advanced class 3B and class 4 heart failure, acute decompensated heart failure, cardio renal syndrome defined by biventricular failure, decreased glomerular filtration rate and systemic congestion, as well as acute coronary syndromes and microvascular angina. These compositions and methods have the possibility to reduce symptoms, reduce hospitalizations and increase the quality of life for patients with these conditions. In preferred embodiments the compositions are administered by continuous intravenous infusion which may be combined with standard therapies.

In another embodiment the patient suffers from a disease selected from the group consisting of myocardial infarct, acute coronary syndrome, unstable angina, non-Q-wave cardiac necrosis, Q-wave myocardial infarct and morbidity after stroke.

In another embodiment, the patient having the cardiovascular disease is a diabetic patient. In yet another embodiment, the patient having the cardiovascular disease is a non-diabetic patient.

The methods and compositions of the present invention may be used to provide acute cardioprotective effects, such as reducing the incidence of sudden death due to arrhythmias or contractile failure in a subject with an acute occlusion of a coronary artery (myocardial infarction); reducing damage occurring during reperfusion of the heart muscle after ischemia ('hypoxia-reperfusion injury' or ‘ischemia-reperfusion injury’); reducing the amount of cardiac muscle that is damaged or reducing the severity of damage to the heart muscle caused by an acute coronary artery occlusion (often referred to as ‘reducing infarct size’) Chronic cardioprotective effects include, but are not limited to, reducing pathologic remodeling of the cardiac chambers, including chamber dilation, consequent to an acute coronary artery occlusion; reducing apoptosis in cardiac muscle consequent to an acute coronary artery occlusion; reducing the impairment of contractility of cardiac muscle consequent to an acute coronary occlusion; and reducing long-term mortality in subjects have suffered damage to the heart muscle caused by an acute coronary occlusion.

Acute and/or chronic cardioprotective effects can be desirable in subjects with chronic coronary artery disease (in which blood flow to the heart muscle is compromised without an acute coronary occlusion, also referred to as ischemic heart disease), myocarditis, idiopathic dilated cardiomyopathy, hypertrophic cardiomyopathy, restrictive cardiomyopathy, infiltrative cardiomyopathy, valvular heart disease, adult congenital heart disease, toxic cardiomyopathy (including but not limited to doxorubicin-induced cardiomyopathy), hypertensive cardiomyopathy, cardiomyopathy associated with endocrine disease, including diabetes, cardiomyopathy associated with connective tissue disease, cor pulmonale, pulmonary arterial hypertension, pulmonary embolism.

The methods and compositions of the present invention can also have an inotropic effect, increasing the strength of contraction in a failing heart. Acute and chronic inotropic effects may be desirable in acute coronary artery disease, chronic coronary artery disease (in which blood flow to the heart muscle is compromised without an acute coronary occlusion, also referred to as ischemic heart disease), myocarditis, idiopathic dilated cardiomyopathy, hypertrophic cardiomyopathy, restrictive cardiomyopathy, infiltrative cardiomyopathy, valvular heart disease, adult congenital heart disease, toxic cardiomyopathy (including but not limited to doxorubicin-induced cardiomyopathy), hypertensive cardiomyopathy, cardiomyopathy associated with endocrine disease, including diabetes, cardiomyopathy associated with connective tissue disease, cor pulmonale, pulmonary arterial hypertension, pulmonary embolism.

The methods and compositions of the present invention may also have an anti-arrhythmic effect. This effect can be acute or chronic, and can include effects that are attributable to prevention and/or reduction of injury to the heart muscle. Examples of anti-arrthymic effects include, but are not limited to, reducing the incidence and altering the rates of cardiac arrhythmias (including but not limited to atrial fibrillation, other supraventricular arrhythmias, ventricular tachycardia and ventricular fibrillation) following coronary occlusion.

The methods and compositions of the present invention may also have an anti-hypertrophic effect. Anti-hypertrophic effects can be desirable in subjects with acute coronary artery disease, chronic coronary artery disease (in which blood flow to the heart muscle is compromised without an acute coronary occlusion, also referred to as ischemic heart disease), myocarditis, idiopathic dilated cardiomyopathy, hypertrophic cardiomyopathy, restrictive cardiomyopathy, infiltrative cardiomyopathy, valvular heart disease, adult congenital heart disease, toxic cardiomyopathy (including but not limited to doxorubicin-induced cardiomyopathy), hypertensive cardiomyopathy, cardiomyopathy associated with endocrine disease, including diabetes, cardiomyopathy associated with connective tissue disease, cor pulmonale, pulmonary arterial hypertension, pulmonary embolism.

The methods and compositions of the present invention can also have lusitropic effects, improving the relaxation of the heart muscle during diastole. Lusitropic effects can be desirable in subjects with acute coronary artery disease, chronic coronary artery disease (in which blood flow to the heart muscle is compromised without an acute coronary occlusion, also referred to as ischemic heart disease), myocarditis, idiopathic dilated cardiomyopathy, hypertrophic cardiomyopathy, restrictive cardiomyopathy, infiltrative cardiomyopathy, valvular heart disease, adult congenital heart disease, toxic cardiomyopathy (including but not limited to doxorubicin-induced cardiomyopathy), hypertensive cardiomyopathy, cardiomyopathy associated with endocrine disease, including diabetes, cardiomyopathy associated with connective tissue disease, cor pulmonale, pulmonary arterial hypertension, pulmonary embolism.

The methods and compositions of the present invention can also have anti-arrhythmic effects of benefit in the treatment of disorders of the heart rhythm, examples of which include but are not limited to atrial fibrillation, ventricular tachycardia and ventricular fibrillation. These effects, which can include reductions in the incidence and rate of the arrhythmias, can be desirable in subjects with acute coronary artery disease, chronic coronary artery disease (in which blood flow to the heart muscle is compromised without an acute coronary occlusion, also referred to as ischemic heart disease), myocarditis, idiopathic dilated cardiomyopathy, hypertrophic cardiomyopathy, restrictive cardiomyopathy, infiltrative cardiomyopathy, valvular heart disease, adult congenital heart disease, toxic cardiomyopathy (including but not limited to doxorubicin-induced cardiomyopathy), hypertensive cardiomyopathy, cardiomyopathy associated with endocrine disease, including diabetes, cardiomyopathy associated with connective tissue disease, cor pulmonale, pulmonary arterial hypertension, pulmonary embolism.

The patient treated using the methods and compositions of the present invention can also be at an increased risk of developing heart disease. This can include (but is not limited to) individuals with hypertension (systemic or pulmonary), obesity, endocrine disease (including diabetes, thyroid disease, adrenal disease, dysregulation of homocysteine metabolism), iron storage disease, amyolidosis, renal disease, connective tissue disease, infectious diseases, thromboembolic disease, immune diseases, hematologic diseases.

Provided herein are methods of increasing or enhancing the chances of survival of a subject with heart disease, comprising administering to a subject in need thereof an effective amount of a GLP-1 compound and/or GLP-1 metabolite of the invention, thereby increasing or enhancing the chances of survival of the subject treated by a certain period of time, for example, by at least 10 days, 1 month, 3 months, 6 months, 1 year, 1.5 years, 2 years, 3 years, 4 years, 5 years, 8 years, or 10 years. The increase in survival of a subject can be defined, for example, as the increase in survival of a preclinical animal model by a certain period of time, for example, by at least 10 days, 1 month, 3 months, 6 months, or 1 year, or at least 2 times, 3 times, 4 times, 5 times, 8 times, or 10 times, more than a control animal model (that has the same type of disease) without the treatment with the inventive method. Optionally, the increase in survival of a mammal can also be defined, for example, as the increase in survival of a subject with heart disease by a certain period of time, for example, by at least 10 days, 1 month, 3 months, 6 months, 1 year, 1.5 years, 2 years, 3 years, 4 years, 5 years, 8 years, or 10 years more than a subject with the same type of heart disease but without the treatment with the inventive method. The control subject may be on a placebo or treated with supportive standard care such as chemical therapy, biologics and/or radiation that do not include the inventive method as a part of the therapy.

GLP-1 metabolites of the invention can be formulated and administered to a subject, are now described. The invention encompasses the preparation and use of pharmaceutical compositions comprising a composition useful for the treatment of a disease or disorder, including, but not limited to diabetes, insulin insufficiency, obesity and glycemic dysregulation. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. As used herein, the term “pharmaceutically-acceptable carrier” means a chemical composition with which an appropriate peptide composition, may be combined and which, following the combination, can be used to administer the appropriate peptide composition to a subject.

The pharmaceutical compositions useful for practicing the invention may be administered to deliver a dose of between about 0.1 ng/kg/day and 100 mg/kg/day. In various embodiments, the pharmaceutical compositions useful in the methods of the invention may be administered, by way of example, systemically, parenterally, orally, or topically. In addition to the appropriate therapeutic composition, such pharmaceutical compositions may contain pharmaceutically acceptable carriers and other ingredients known to enhance and facilitate drug administration.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation.

Pharmaceutical compositions that are useful in the methods of the invention may be prepared, packaged, or sold in formulations suitable for oral, parenteral, topical, intravenous, intramuscular, and other known routes of administration.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, cutaneous, subcutaneous, intraperitoneal, intravenous, and intramuscular.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g., sterile pyrogen-free water) prior to parenteral administration of the reconstituted composition.

Typically dosages of the compound of the invention which may be administered to an animal, preferably a human, range in amount from about 0.01 mg to 20 about 100 g per kilogram of body weight of the animal. While the precise dosage administered will vary depending upon any number of factors, including, but not limited to, the type of animal and type of disease state being treated, the age of the animal and the route of administration. Preferably, the dosage of the compound will vary from about 1 mg to about 100 mg per kilogram of body weight of the animal. More preferably, the dosage will vary from about 1 μg to about 1 g per kilogram of body weight of the animal. The compound can be administered to an animal as frequently as several times daily, or it can be administered less frequently, such as once a day, once a week, once every two weeks, once a month, or even less frequently, such as once every several months or even once a year or less. The effects of continuous intravenous infusion of GLP-1 metabolites are not acute but rather seen over a 72 hour period. Therefore, these agents, like the native peptide, are preferably administered continuously for prolonged periods. The frequency of the dose will be readily apparent to the skilled artisan and will depend upon any number of factors, such as, but not limited to, the type and severity of the disease being treated, the type and age of the animal, etc.

It will be appreciated by one of skill in the art, when armed with the present disclosure including the methods detailed herein, that the invention is not limited to treatment of a disease or disorder that is already established. Particularly, the disease or disorder need not have manifested to the point of detriment to the subject; indeed, the disease or disorder need not be detected in a subject before treatment is administered. That is, significant disease or disorder does not have to occur before the present invention may provide benefit. Therefore, the present invention includes a method for preventing a disease or disorder in a subject, in that a peptide composition, as discussed elsewhere herein, can be administered to a subject prior to the onset of the disease or disorder, thereby preventing the disease or disorder. The preventive methods described herein also include the treatment of a subject that is in remission for the prevention of a recurrence of a disease or disorder.

One of skill in the art, when armed with the disclosure herein, would appreciate that the prevention of a disease or disorder encompasses administering to a subject a peptide composition as a preventative measure against the disease or disorder.

The invention encompasses administration of a peptide to practice the methods of the invention; the skilled artisan would understand, based on the disclosure provided herein, how to formulate and administer the appropriate peptide to a subject. Indeed, the successful administration of the peptide has been reduced to practice as exemplified herein. However, the present invention is not limited to any particular method of administration or treatment regimen.

### Dosage

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds ties preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a haft-maximal inhibition of symptoms) as determined in cell culture. Such information can be used to more accurately determine useful doses in humans. Levels in plasma may be measured, for example, high performance liquid chromatography.

### Combination Therapy

The preventive or therapeutic compositions of the present invention can also be used in combination with conventional therapeutics of heart failure such as diuretics, inotropes, coronary vasodilators and beta blockers or conventional therapeutics of circulatory diseases such as hypertension (e.g. angiotensin converting enzyme (ACE) inhibitors, angiotensin receptor blockers (ARBs) and/or calcium channel blockers), either simultaneously or at different times. Diuretics are generally used for relief of congestive symptoms and help the kidneys rid the body of excess fluid, thereby reducing blood volume and the heart's workload. Diuretics can include, but are not limited to loop diuretics (e.g. furosemide, bumetanide); thiazide diuretics (e.g. hydrochlorothiazide, chlorthalidone, chlorthiazide); potassium-sparing diuretics (e.g. amiloride); spironolactone and eplerenone. Inotropes, such as a cardiac glycoside, a beta-adrenergic agonist or a phosphodiesterase inhibitor, strengthen the heart's pumping action in patients with low cardiac output; inotropes can include but are not limited to digoxin, dobutamine, milrinone, istaroxime, omecamtiv mecarbil. Vasodilators, cause the peripheral arteries to dilate, making it easier for blood to flow; examples of vasodilators include, but are not limited, nitroglycerin, nitorprusside, and neseritide. Activation of neurohormonal systems that include the renin-andiotensin-aldosterone system (RAAS) and the sympathetic nervous system also contribute to the pathophysiology of heart failure. Drugs that inhibit activation of RAAS fall into three major categories: ACE inhibitors (including but not limited to ramipril, enalapril, and captopril), ARBs (including but not limited to valsarten, candesarten, irbesarten and losarten), and aldosterone receptor blockers (e.g., spironolactone and eplerenone.) Beta blockers counter the effects of activation of the sympathetic nervous system and slow the heart rate by blocking the effects of adrenalin; beta blockers include, but are not limited to carvedilol, metoprolol, bisoprolol, atenolol, propranolol, timolol and bucindolol.

### Kits

The present invention also pertains to kits useful in the methods of the invention. Such kits comprise various combinations of components useful in any of the methods described elsewhere herein, including for example, a GLP-1 metabolite, materials for quantitatively analyzing a GLP-1 metabolite, materials for assessing the activity of a GLP-1 metabolite, materials for assessing the treatment of a disease or disorder by administrating of a GLP-1 metabolite, and an instructional material. For example, in one embodiment the kit comprises components useful for the assessment of the activity a GLP-1 metabolite in a patient.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### Cardio-Metabolic and Vascular Effects of GLP-1 Metabolites

The salutary cardiovascular effects of native GLP-1 (7-36) amide and its active metabolite, GLP-I (9-36) amide have been described (U.S. Pat. No. 7,192,922). However, the results presented herein are directed to improvements on GLP-1 based therapy in view of the FDA requirement for cardiovascular outcomes associated with approval of all new agents in the treatment of type 2 diabetes. The results presented herein demonstrate proof of concept in both animal models (mice, rats, dogs, pigs) and humans demonstrating salutary cardiovascular effects in post-ischemia models and models of heart failure.

Recently, two distinct cellular mechanisms by which GLP-1 mitigates post-ischemic injury as well as direct renal protective effects were discovered. Notably, the native peptide, GLP-1 (7-36) is rapidly degraded by DPP-4 to GLP-1 (9-36) which is then further metabolized by NEP2.1 to a nano GLP-1 (28-36) and pentapeptide, GLP-I (32-36). Virtually, all of the prior work focused on the use of GLP-1 in CV diseases had employed the same long acting DDP-4 resistant peptides that have been approved for clinical use in diabetes. These approaches attempted to obviate the short circulatory half-life of native GLP-1 to maximize its interaction with GLP-1 receptors on pancreatic beta cells and other therapeutic targets including the myocardium. However, the results presented herein demonstrate that the first metabolite, GLP-I (9-36) had similar CV effects as GLP-1 (7-36) when infused in conscious, chronically instrumented dogs with pacing induced heart failure. This led to the hypothesis that there were both GLP-1 receptor dependent and receptor independent mechanisms of CV actions of GLP-1. Without wishing to be bound by any particular theory, it is believed that the CV benefits may be enhanced by preparations which allow for endogenous cleavage and processing of GLP-1 (7-36) into its metabolites which then confer additional CV benefits, including insulin independent increases in myocardial glucose uptake and systemic, coronary, and renal vasodilation.

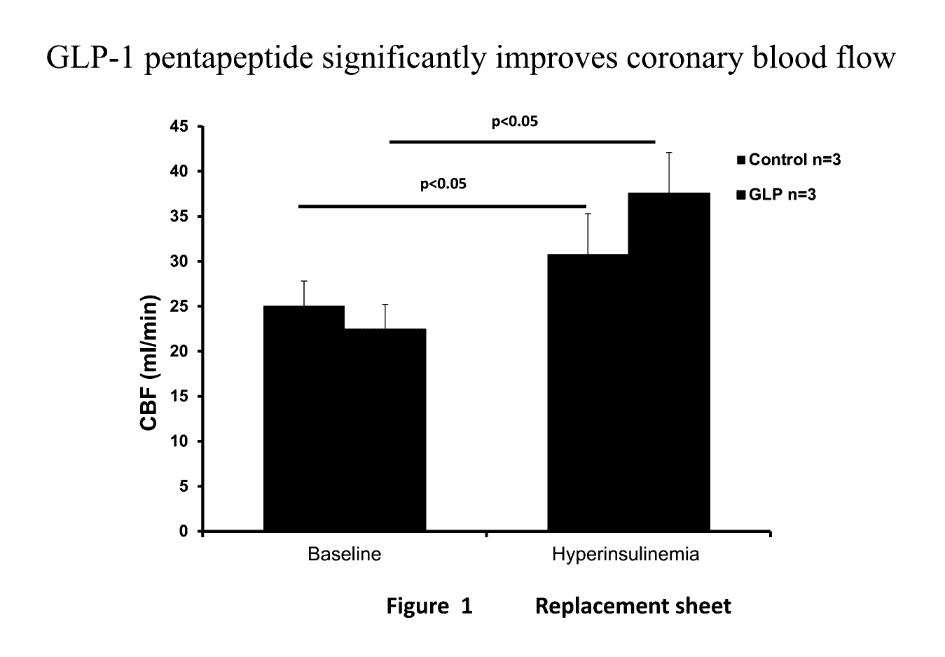

To determine the effect of GLP-1 (32-36) on coronary blood flow, hyperglycemic clamp studies were conducted in normal, conscious, chronically instrumented dogs (n=3), in which plasma glucose was raised to 180 mg/dl by continuous infusion of glucose (FIG. 1). The plasma glucose concentration was monitored continuously as was plasma insulin response. In the control studies, the 5 dogs underwent the 3 hour hyperglycemic clamp. On a separate occasion, the same animals underwent a 3 hour hyperglycemic clamp in which GLP-1 (32-36) was infused beginning in the second hour. A significantly greater increase in coronary blood flow was observed in the presence of GLP-1 (32-36) than was observed under the control circumstances.

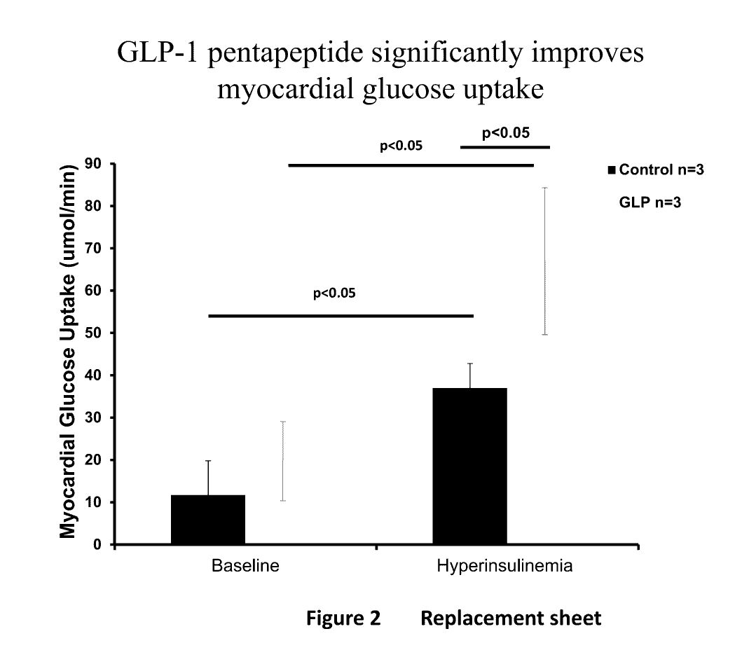

Similarly, in FIG. 2, a significantly greater increase in myocardial glucose uptake in the presence of GLP-1 (32-36) compared to the control was observed. Myocardial glucose uptake is calculated as the product of the difference in the arterial-coronary sinus glucose content and the average coronary blood flow. The increase in myocardial glucose uptake is in excess of that attributable to the increase in coronary blood flow alone. Taken together, these data suggest that the pentapeptide GLP-1 (32-36) has coronary vasodilator and glucostimulatory effects on the myocardium

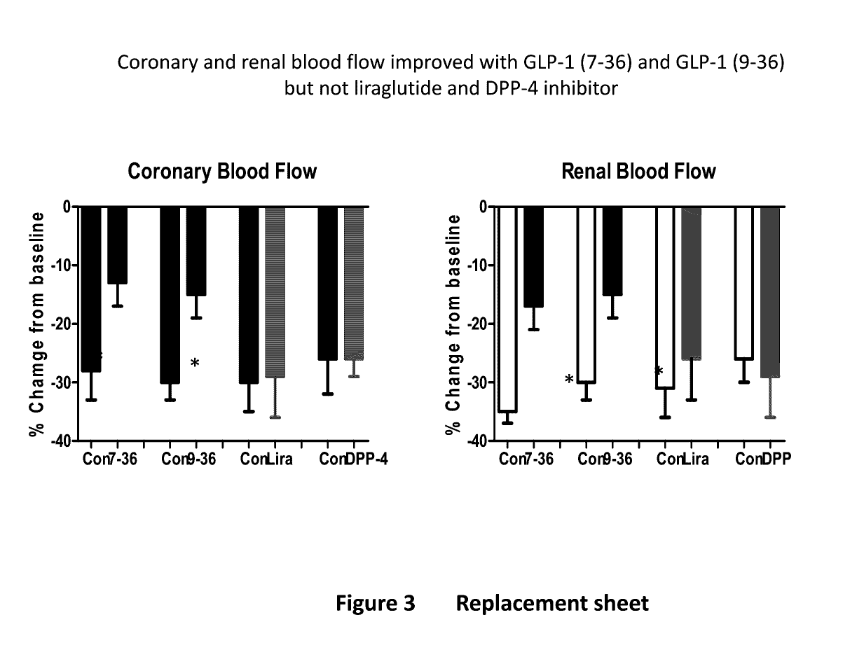

In order to compare the vasoactive effects of different classes of incretins, experiments were designed to study the pacing induced model of dilated cardiomyopathy in conscious chronically instrumented dogs (FIG. 3). These animals were instrumented to examine the effects of equal concentrations of various classes of incretins on coronary blood flow and renal blood flow. All dogs underwent 72 hours of rapid ventricular pacing following which coronary flow responses were measured (Control). Rapid pacing was associated with 30-35% declines in coronary blood flow and renal blood flow respectively. Notably, treatment with either GLP-1 (7-36) amide or GLP-1 (9-36) amide preserved coronary and renal blood flow to a greater extent than seen in control. Notably, treatment with the GLP-1 analogue liraglutide which is DPP-4 resistant and therefore does not generate GLP-1 (9-36) did not preserve coronary or renal blood flow compared to Control. Similarly, the DPP-4 inhibitor saxagliptin did not preserve coronary or renal blood flow. Taken together, these data suggest that native GLP-1 (7-36) amide and the active metabolite GLP-1 (9-36) amide preserve coronary and renal blood flow following rapid pacing when other incretin based therapies do not. This supports the notion that the native peptide and active metabolites have vasoactive as well as glucose-stimulatory properties. The renal vascular effects support the claims for use in heart failure complicated by renal failure (cardio-renal syndrome). The coronary vasodilator properties support the claims for the use in acute coronary syndromes and microvascular angina.

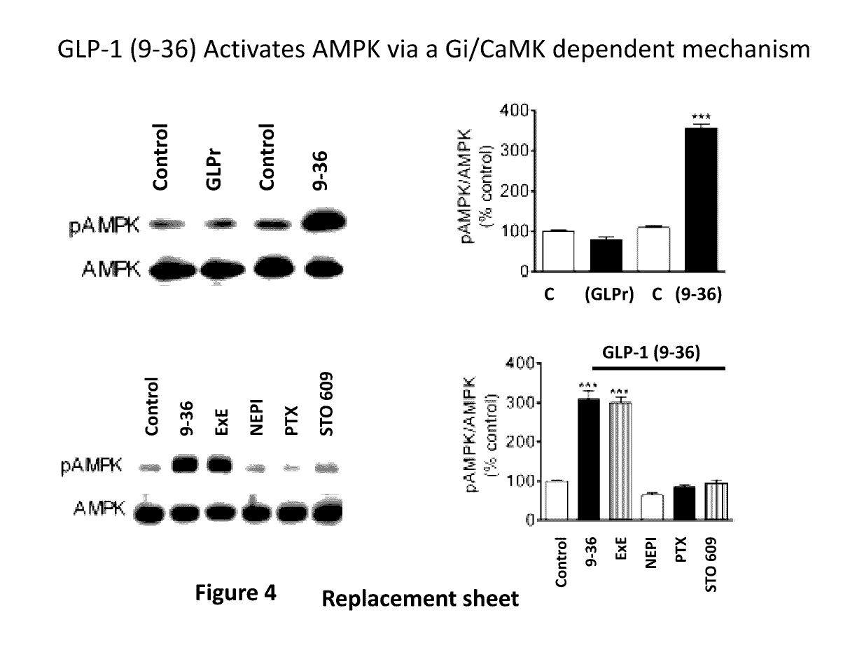

It is largely unknown how GLP-1 stimulates glucose uptake independent of insulin. A candidate mechanism involves the activation of AMP kinase which causes GLUT translocation and glucose uptake independent of insulin. The isolated isovolumic rat heart was studied as a model in which myocardial specific responses can be observed (FIG. 4). In the upper panel, the hearts were exposed to separate 60 minutes of perfusion with control solution (Control), exenatide (GLPr) and the active metabolite GLP-1 (9-36), respectively. GLP-1 9-36 increased the phosphorylation and the activation of AMP kinase which increases glucose uptake independent of insulin. In the lower panel, it was observed that the effects of GLP-1 (9-36) were not blocked by EXE, which inhibits activation of the GLP-1 receptor indicating that the activation of AMPK was not mediated through the receptor. However, the effects of (9-36) to activate AMPK were abolished by neuroendopeptidase inhibition (NEPI), indicating that the effect was mediated by GLP-1 (32-36). Moreover, the effect was mediated through a Gᵢ coupled mechanism as it was blocked by pertussus toxin (PTX) and involved Ca⁺⁺—Calmodulin kinase 2 (CAM kinase) as it was blocked by ST0609. Taken together, these data suggest that GLP-1(9-36) can activate AMP kinase, independent of the GLP-1 receptor and that the effect is mediated by the pentapeptide GLP-1 (32-36).

These data support the novel concept that the generation of metabolites from the native peptide (GLP-1 7-36 amide) is an activation process, not a degradation process, and that vasodilation and glucose uptake is mediated through these activation steps.

The results presented herein demonstrate that GLP-1 (9-36) mediates the systemic, coronary, and renal vasodilator effects of incretins and that these effects are unrelated to GLP-1 receptor activation and therefore not observed with GLP-1 receptor agonists or DDP-4 inhibitors. In addition, the results demonstrate that the pentapeptide GLP-1 (32-36) mediates increases in coronary blood flow and myocardial glucose uptake in a GLP-1 receptor independent manner. Furthermore, the results demonstrate that the mechanism whereby GLP-1 (32-36) amide stimulates myocardial glucose uptake is via activation of AMP kinase, and energy sensor that regulates cell survival.

Several GLP-1 analogues are commercially available. However, without wishing to be bound by any particular theory it is believed that there are unique benefits associated with the use of the native peptide, GLP-1 (7-36) amide and that these benefits are attributable to the active metabolites, GLP-1 (9-36) amide and GLP-1 (32-36). Preferably, these peptides are administered to a mammal by continuous infusion.

The metabolites of the invention can be readily synthesized. The synthesized agent does not cause hypoglycemia in non-diabetic subjects. In addition, the results indicate that the metabolites of GLP-1 (7-36) amide [GLP-1 (9-36) amide and GLP-1 (32-36) amide] are active in both stimulating glucose uptake into myocardium in a non-insulin dependent fashion and in preconditioning donor organs against ischemic injury. Most pharmaceutical companies are focusing on the development of long acting analogues of GLP-1 that are DPPIV resistant. While these agents may be effective in stimulating pancreatic insulin release, they may not be as effective in stimulating myocardial glucose uptake or in mediating vasodilation which are critical determinants of favorable outcomes in CV disease. Moreover, the results presented herein depict the mechanism of action in activating AMPK, a key cellular sensor of energetic balance in cardiomyocytes.

The effects of continuous intravenous infusion of GLP-1 metabolites are not acute but rather seen over a 2-6 hour period. Therefore, these agents, like the native peptide, are preferably administered continuously for prolonged periods to achieve optimal benefits. The effects of the metabolites also wane when the infusion is discontinued.

It is believed that the present invention is useful to decrease mortality and re-hospitalizations in acute decompensated heart failure and similarly in cardio-renal syndrome where the combination of heart failure and renal failure predicts the highest rates of mortality.

### Example 2

### Native GLP-1 and its Active Metabolites in the Treatment of Cardiovascular Disease

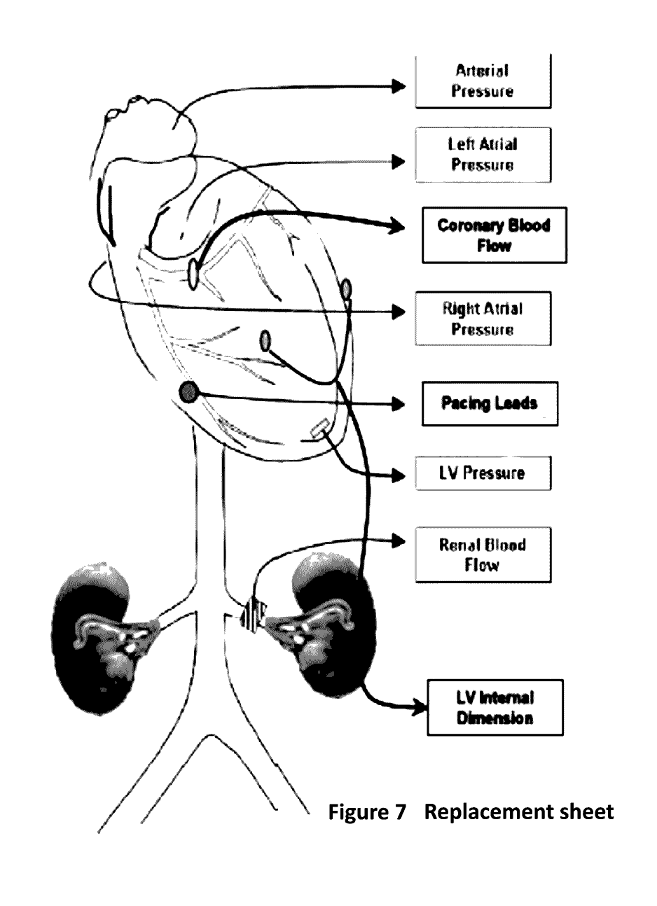

FIG. 7 illustrates characteristic instrumentation of conscious, chronically instrumented dogs studied in the laboratory. Under sterile surgical technique, the animals are instrumented as indicated and allowed to recover fully from their surgery. LV, aortic, and left atrial catheters were used to measure pressures. LV Konigsberg® transducer was used to measure isovolumic contractility and relaxation as measures of systolic and diastolic function. Ultrasonic dimension crystals were used to measure LV shortening and LV filling rates. Pacing leads were attached to the left atrial appendage. Transonics® flow probes were placed around the left circumflex coronary artery and right renal artery to measure coronary and renal blood flow.

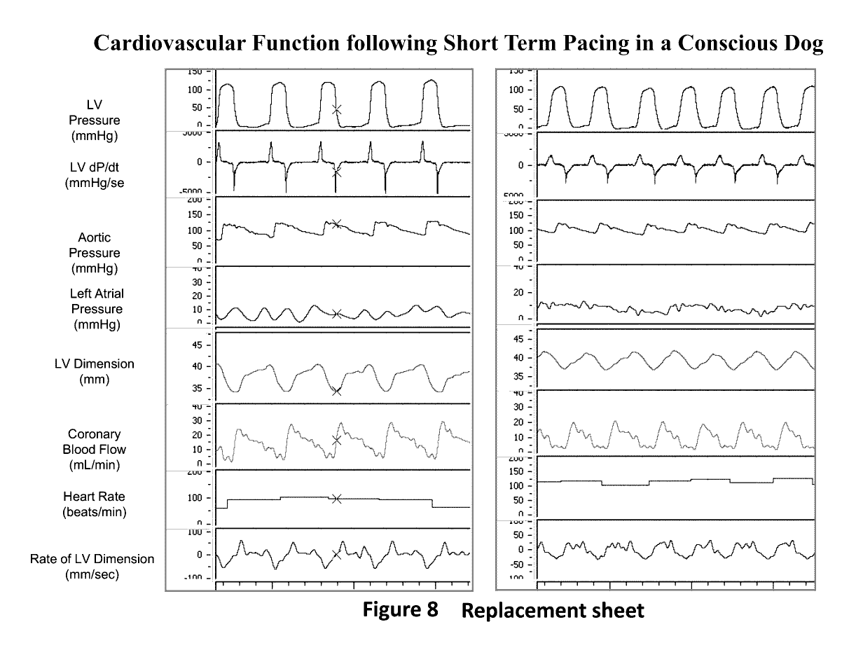

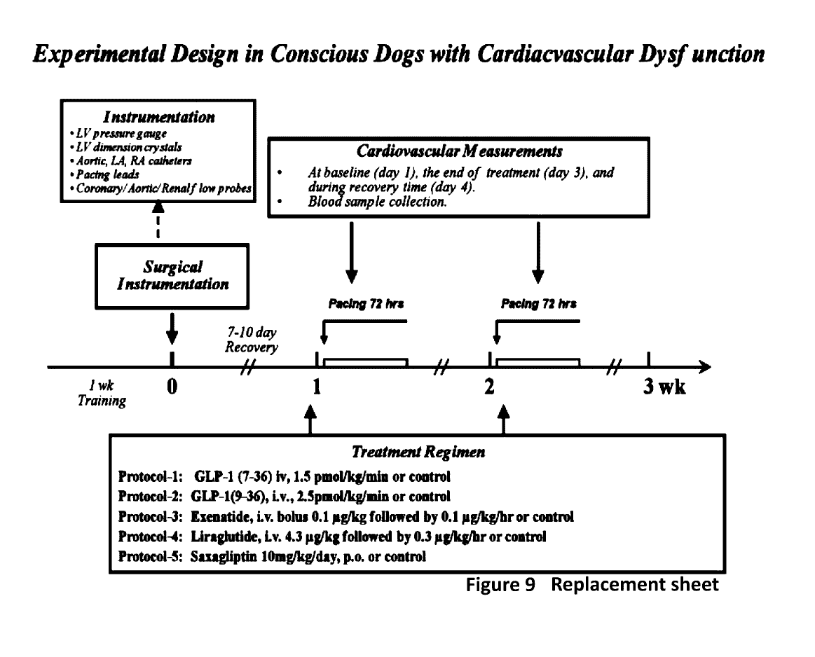

Experiments were performed where 25 conscious, chronically instrumented dogs (FIG. 7) were subjected to a rapid pacing induced stress which recapitulates the hemodynamic perturbations experienced by patients with sudden onset of paroxysmal atrial fibrillation (FIG. 8). Dogs were studied before and daily during the 72 hour pacing stress. Each dog served as its own control with sham and active treatment arms randomized and separated by one week after it was confirmed that hemodynamics returned to baseline levels following the cessation of the pacing stimulus. Dogs received either a sham vehicle infusion or the following (FIG. 9):
GLP-1 (7-36) amide at 5 ng/kg/min IV infusion
GLP-1 (9-36) amide at 10 ng/kg/min IV infusion
Liraglutide 4.3 μg/kg IV bolus followed by 0.3 μg/kg/hr IV infusion
Exenatide 0.1 μg/kg IV bolus followed by 0.1 μg/kg/hr IV infusion
Saxagliptin 10mg/kg/day, p.o. daily
   Hemodynamic measurements were recorded at baseline and at 72 hours following the rapid pacing stress. All measurements were made during intrinsic sinus rhythm 20-30 minutes after cessation of pacing. Hemodynamic measurements included:
LV systolic and diastolic pressures, mean arterial pressures, heart rate
Systolic function: LV contractility (systolic contractile function), LV fractional shortening
Diastolic function: LV isovolumic relaxation time constant, LV filling rate
Flows: systemic, coronary and renal blood flows

The results of the experiments are summarized in FIG. 8. Three days of rapid pacing was associated with:
12 mmhg reduction in LV systolic pressure and 15 mmHg reduction in mean arterial pressure
5 mmHg increase in LV end diastolic pressure
45% reduction in LV systolic contractile function
30% reduction in LV fractional shortening
40% reduction in LV filling rate and a 50% reduction in the isovolumic relaxation time constant
38% reduction in coronary blood flow
20% reduction in renal blood flow
335 increase in systemic vascular resistance

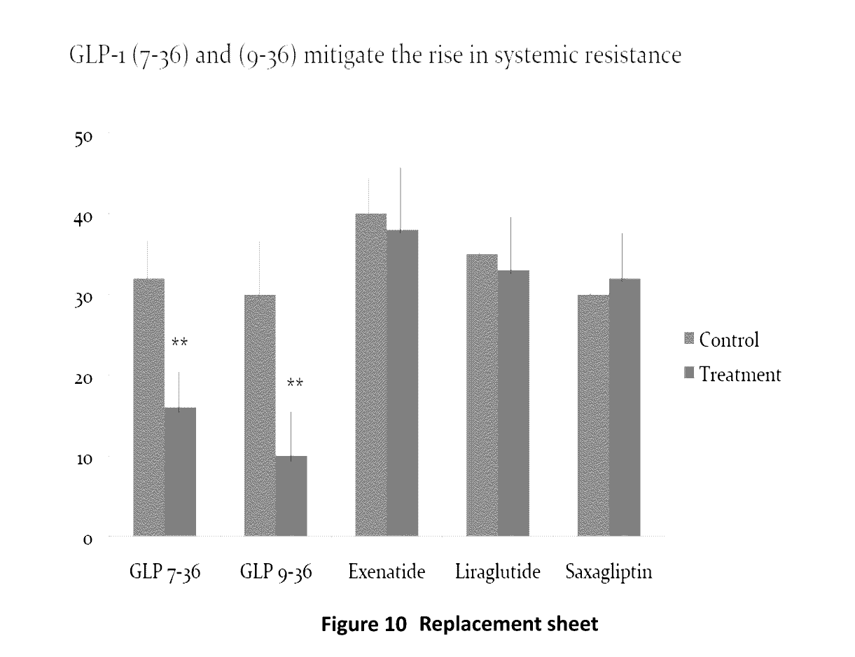

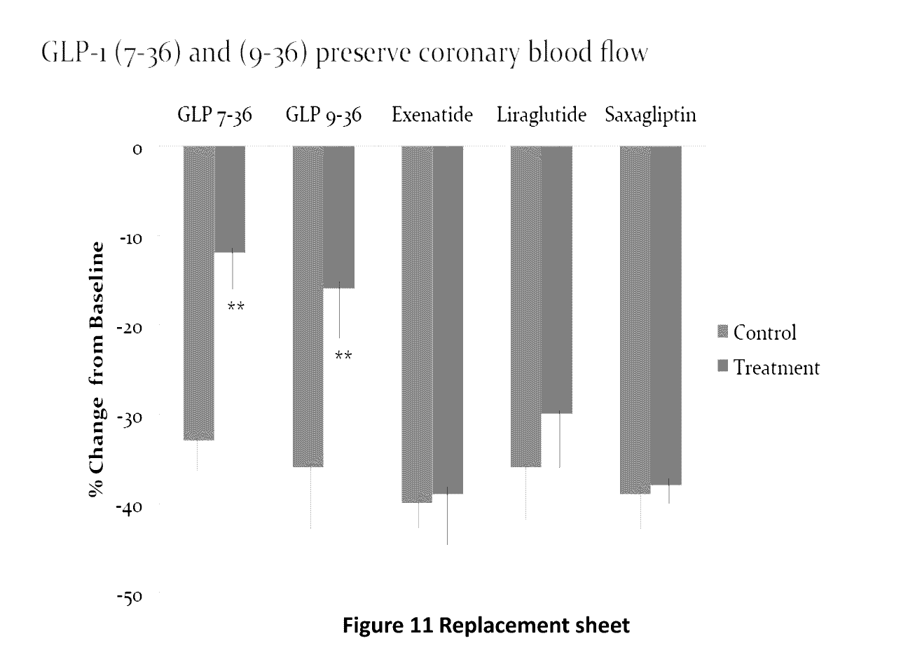

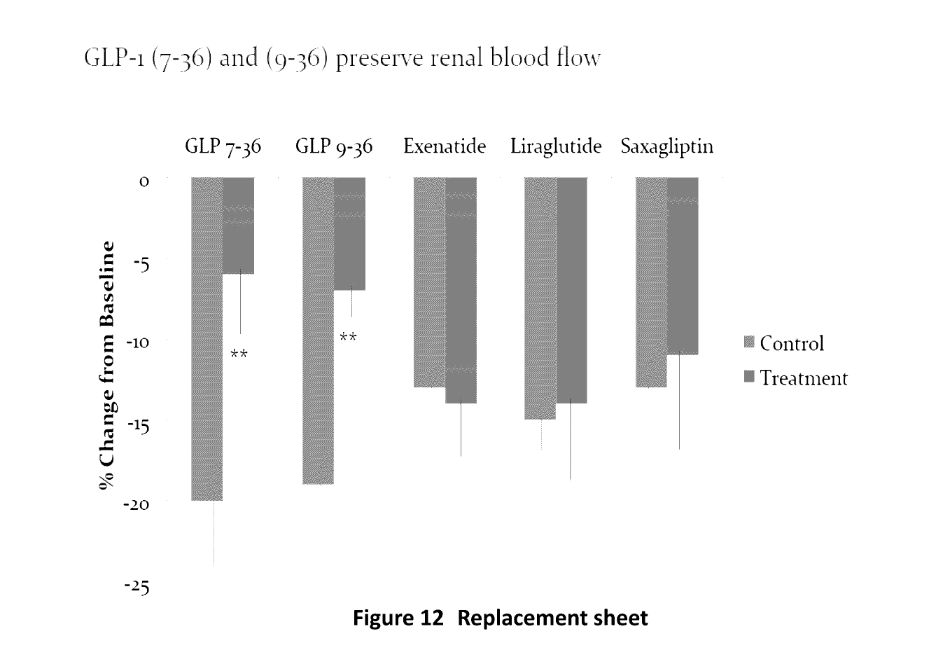

The results presented herein demonstrate that both the native peptide, GLP-1 7-36 amide and its active metabolite GLP-1 9-36 amide, mitigated the declines in LV systolic and diastolic function and the rise is systemic vascular resistance (FIG. 10) preserved coronary (FIG. 11) and renal blood flow (FIG. 12). However, liraglutide and exenatide, which are GLP-1 receptor agonist that are resistant to DPPIV and are not metabolized to the active metabolite, had no effect on LV systolic or diastolic function or on systemic, coronary or renal blood flow. Similarly, saxagliptin, which is a DPP-4 inhibitor, had no effect.

These data demonstrate that the native peptide and its active metabolite possess beneficial cardiovascular properties that are salutary in preserving cardiovascular function and regional and system blood flow during a relevant CV stress such as that imposed by paroxysmal atrial fibrillation. These findings support the use of native GLP-1 and its active metabolites in the treatment of cardiovascular disease.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method for treating a cardiovascular disease in a patient, the method comprising administering to a patient in need thereof a GLP-1 selected from the group consisting of GLP-1 (32-36) amide, and pharmaceutically-acceptable salts thereof, and any combination thereof.

2. The method of claim 1, wherein the cardiovascular disease is advanced heart failure.

3. The method of claim 2, wherein the advanced heart failure is selected from the group consisting of class 3B heart failure and class 4 heart failure.

4. The method of claim 1, wherein the cardiovascular disease is decompensated heart failure.

5. The method of claim 1, wherein the cardiovascular disease is cardio renal syndrome.

6. The method of claim 5, wherein the cardio renal syndrome is defined by biventricular failure, decreased glomerular filtration rate, and systemic congestion.

7. The method of claim 1, wherein the cardiovascular disease is acute coronary syndrome.

8. The method of claim 1, wherein the cardiovascular disease is microvascular angina.

9. The method of claim 1, wherein the cardiovascular disease is symptomatic heart failure with preserved ejection fraction.

10. The method of claim 1, wherein the cardiovascular disease is angina pectoris and ventricular hypertrophy that accompany Friedreich's ataxia.

11. The method of claim 1, wherein the GLP-1 is administered intravenously.

12. The method of claim 1, wherein the GLP-1 is administered by continuous intravenous infusion.

13. The method of claim 1, wherein the GLP-1 is administered at a rate of 1.25-10 pmol/kg/min by continuous intravenous infusion.

14. The method of claim 11, wherein the continuous intravenous infusion is for at least 72 hours.

15. A composition comprising GLP-1 (32-36) amide and a pharmaceutically acceptable salt thereof.

16. The composition of claim 15, further comprising a GLP metabolite selected from the group consisting of GLP-1 (9-36) amide, GLP-1 (7-36) amide, and a combination thereof.

17. The method of claim 1, wherein the method further comprises administering a second GLP-1 selected from the group consisting of GLP-1 (7-36) amide, GLP-1 (9-36) amide, and pharmaceutically-acceptable salts thereof, and any combination thereof.


SEQUENCE LISTING (4 sequences)

SEQ ID NO: 1 - protein, 31 aa, Artificial Sequence
  Chemically synthesized
HAEGTFTSDVSSYLEGQAAKEFIAWLVKGRG

SEQ ID NO: 2 - protein, 30 aa, Artificial Sequence
  Chemically synthesized
HAEGTFTSDVSSYLEGQAAKEFIAWLVKGR

SEQ ID NO: 3 - protein, 28 aa, Artificial Sequence
  Chemically synthesized
EGTFTSDVSSYLEGQAAKEFIAWLVKGR

SEQ ID NO: 4 - protein, 5 aa, Artificial Sequence
  Chemically synthesized
LVKGR